(19) United States
(12) Patent Application Publication
(10) Pub. No.: US 2016/0184533 A1
(43) Pub. Date: Jun. 30, 2016

(54) MECHANISM FOR A DRUG DELIVERY DEVICE

(71) Applicant: SANOFI SA, Vernier (CH)

(72) Inventor: Stefan MAYER, Frankfurt am Main (DE)

(73) Assignee: Sanofi SA, Vernier (CH)

(21) Appl. No.: 14/892,308

(22) PCT Filed: Apr. 26, 2014
(86) PCT No.: PCT/EP2014/058386

§ 371 Date: Nov. 19, 2015

(30) Foreign Application Priority Data
Apr. 26, 2013 (EP) 13165599.5

(51) Int. Cl. A61M 5/50; A61M 15/00; A61M 5/315

(52) U.S. Cl. CPC A61M 5/50; A61M 5/31533; A61M 15/0001; A61M 2205/273; A61M 2202/064

(57) ABSTRACT

A mechanism for a drug delivery device, having a first and a second state, including an engagement preventing member, and a blocking member having a blocking feature and an engagement element which is engagable with the blocking member. The blocking member is configured to engage with the engagement element in a non-linear movement, thereby moving the blocking feature is moved from a first position to a second position in an arc shaped movement. In the first state of the mechanism, the engagement preventing member is configured to prevent an engagement of the blocking member with the engagement element by covering the engagement element, and wherein, in the second state of the mechanism, the engagement element is free from the engagement preventing member such that the blocking member is enabled to engage with the engagement element.

This disclosure relates to a mechanism for a drug delivery device. The drug delivery device may be an inhalator, in particular a dry powder inhaler. An inhalation device is usually activated by the user's suction airstream and is intended for the inhalation of a substance, in particular a powdery substance. An inhalation device is described in document WO 2009/065707 A1, for example.

However, the mechanism is also suitable for other drug delivery devices, e.g. an injector such as an injection pen. In particular, the mechanism may be used in a fixed-dose drug delivery device, i.e. a device in which the size of the drug which is delivered may not be varied by the user. Rather, in a fixed-dose device the size of the dose is set by the design of a dispensing mechanism.

It is an object of the present disclosure to provide an improved mechanism for a drug delivery device, i.e. a small and reliable mechanism.

This object may, inter alia, be achieved by the subject-matter of the independent claim. Advantageous embodiments and refinements are the subject-matter of the dependent claims. However, further advantageous concepts may be disclosed herein besides the ones which are claimed.

One aspect of the present disclosure relates to a mechanism for a drug delivery device having a first and second state and comprising an engagement preventing member, a blocking member comprising a blocking feature and an engagement element which is engageable with the blocking member, wherein the blocking member is configured to engage with the engagement element in a non-linear movement, thereby moving the blocking feature from a first position to a second position, wherein, in the first state of the mechanism, the engagement preventing member is configured to prevent an engagement of the blocking member with the engagement element by covering the engagement element, and wherein, in the second state of the mechanism, the engagement element is free from the engagement preventing member such that the blocking feature is enabled to engage with the engagement element.

The mechanism may be a lock-out mechanism which is configured to prevent an operation of the drug delivery device after a certain number of doses has been delivered. The mechanism may also be a counting mechanism configured to count and display the number of doses that have been delivered by the drug delivery device. Alternatively, the counting mechanism may be configured to count and to display the number of doses that are left in the drug delivery device before the drug delivery device is considered empty. Moreover, the mechanism may be a combined lock-out mechanism and counting mechanism. In this case, the mechanism is configured to count the number of dose dispensing operations performed by the drug delivery device and to prevent a further operation of the drug delivery device after a certain number of operations has been carried out. Moreover, the combined mechanism may also display the number of doses left in the device.

The mechanism may be formed by a separate unit which may be engaged with the drug delivery device. For example, the mechanism may be configured to be snap-fitted to the drug delivery device.

The first state of the mechanism may be defined by the engagement preventing member covering the engagement element. Thereby, the engagement preventing member may prevent an engagement of the blocking member with the engagement element. Furthermore, the engagement element thereby ensures that the blocking feature remains in its first position. Accordingly, the first state of the mechanism may also be defined by the blocking feature being in its first position.

The first state of the mechanism may correspond to at least one dose remaining in the drug delivery device. The first state of the mechanism may be defined by the mechanism displaying that at least one dose dispensing operation may be performed. The first state of the mechanism may also be defined by the mechanism enabling an operation of the drug delivery device, e.g. said operation being a dose setting operation and/or a dose dispensing operation.

The second state of the mechanism may be defined by the engagement element being free from the engagement preventing member. Thereby, in the second state of the mechanism, the blocking member may be enabled to engage with the engagement element. Furthermore, in the second state of the mechanism, the blocking feature may be in its second position. When the blocking member is engaged with the engagement element, the blocking member may be configured to block a movement of parts of the mechanism. In particular, the blocking member may be configured to prevent a further movement of a first rotatable member when the blocking member is engaged with the engagement element. Thereby, the blocking member may prevent a further operation of the mechanism. Moreover, the blocking member may be configured to prevent a further operation of the drug delivery device when the blocking member is engaged with the engagement element. The operation may be a dose setting operation and/or a dose dispensing operation.

The non-linear movement by which the blocking member may engage with the engagement element may be a rotation. The non-linear movement may be a helical movement. The blocking feature may move along an arc shaped path, when the blocking member is moved in the non-linear movement. In particular, the blocking feature may be offset from a first rotational axis around which the blocking member is rotated such that the blocking feature is moved in a circular movement.

The engagement preventing member is moveable relative to the engagement element. In particular, the engagement preventing member may be wound around the engagement element in the first state of the mechanism. Furthermore, in the second state of the mechanism, the engagement preventing member may be unwound from the engagement element such that the engagement element is free from the engagement preventing member.

The engagement preventing member may be flexible such that it can be wound and unwound around the engagement element. Preferably, the engagement preventing member comprises a tape. The engagement preventing member may also serve as a counting member configured to count and to display information regarding the drug delivery device. In this case, numbers or symbols may be arranged on the engagement preventing member. In the first state of the mechanism, the engagement preventing member may display the information that at least one dose dispensing operation may be carried out. In the second state of the mechanism, the engagement preventing member may display the information that no further dose dispensing operations can be carried out.

In the second state of the mechanism, the blocking member may be configured to interfere with a dose setting mechanism and/or a dose dispensing mechanism of the drug delivery device. In particular, the blocking feature of the blocking member may engage with an element of the dose setting mechanism and/or a dose dispensing mechanism in the second state of the mechanism such that a further operation of the dose setting mechanism and/or a dose dispensing mechanism of the drug delivery device is prevented.

The blocking feature has a first position and a second position. In its first position, the blocking feature may not interfere with a dose setting mechanism and/or a dose dispensing mechanism of the drug delivery device. In particular, in its first position, the blocking feature may not be engaged with an interaction member. In its first position, the blocking feature may be arranged outside a guiding track along which the interaction member is configured to travel. In its second position, the blocking feature may be engaged with an interaction member, thereby preventing a further operation of the drug delivery device. In its second position, the blocking feature may be arranged in the guiding track along which the interaction member is configured to travel.

The blocking member may be configured to rotate around a first rotational axis. The blocking feature may be moved from its first to its second position in an arc-shaped movement around the axis. In particular, the blocking feature may be arranged offset from the axis. The movement of the blocking feature from its first to its second position may not be a linear movement. The blocking feature may be a projection of the blocking member.

When the mechanism is engaged with the drug delivery device, the first rotational axis may be parallel to a longitudinal axis of the drug delivery device.

In one embodiment, the engagement element may comprise an opening and the blocking member may comprise a protrusion configured to engage with the opening. Accordingly, the opening may be configured to receive the protrusion of the blocking member. When the opening of the engagement element is free from the engagement preventing member in the second state of the mechanism, that the blocking member may be moved, e.g. rotated, such that the protrusion engages with the opening.

When the blocking feature is in the first position, the blocking member may be tensioned to rotate and thereby to move the blocking feature into the second position. Thereby, it is ensured that, once the engagement element is free from the engagement preventing member, the blocking member engages with the engagement element. Without the tension of the blocking member, an additional force would be required to move the blocking member into engagement with the engagement element. As the blocking member is tensioned to rotate, the engagement of the blocking member and the engagement preventing member is triggered reliably and immediately once the engagement element is free from the engagement preventing member.

In particular, the blocking member may comprise a first and a second tension member configured to abut a first and a second rotatable member and thereby to tense the blocking member. The first and the second tension member may comprise flexible arms.

The blocking member may be rotatable around a first rotational axis and the blocking feature may be offset from the first rotational axis in its first and in its second position. Thus, the blocking feature moves along an arc-shaped path when the blocking member is rotated around the first rotational axis. The first rotational axis may be parallel to the longitudinal axis of the drug delivery device when the mechanism is attached to the drug delivery device. The first rotational axis may run through a centre point of the blocking member.

The mechanism may further comprise a first rotatable member comprising the engagement element and being configured to be rotated by movement of the engagement preventing member. The first rotatable member may comprise a wheel. In particular, the engagement preventing member may be wound around the first rotatable member in the first state of the mechanism. As the engagement preventing member is unwound from the first rotatable member, the first rotatable member is thereby rotated. The engagement element on the first rotatable member may be arranged such that, after the last possible dose dispensing operation has been performed and the engagement preventing member has been unwound from the first rotatable member, the engagement element is aligned with the blocking member such that the blocking member can engage with the engagement element.

In one embodiment, the first rotatable member is configured to rotate around a second rotational axis and the second rotational axis may be parallel to the first rotational axis. The second rotational axis may be a symmetry axis of the first rotatable member. The second rotational axis may go through a centre point of the first rotatable member.

The first rotatable member may comprise a first reverse rotation prevention feature which is configured to permit a rotation of the first rotatable member in the first rotational direction and which is configured to prevent a rotation of the first rotatable member in a second rotational direction opposite to the first rotational direction in particular, the first reverse rotation prevention feature may comprise teeth which are shaped to engage with a corresponding pall such that a rotation in the first rotational direction is allowed and a rotation in the second rotational direction is prevented.

Furthermore, the mechanism may comprise a body comprising a pin. The first rotatable member may have an opening configured to receive the pin and may further be configured to rotate around the pin. The pin may have a first end portion, a second end portion and a middle portion arranged between the first and the second end portion. The diameter of the opening and the diameter of the pin may be chosen such that the first rotatable member contacts the pin only in a first and the second end portion. Thereby, the friction between the first rotatable member and the pin is reduced to a minimum. Furthermore, the pin may comprise an engagement feature. When the blocking member engages with the engagement element, the blocking member may simultaneously also engage with the blocking feature of the pin.

Furthermore, the mechanism may comprise a second rotatable member which is configured such that the engagement preventing member is moveable by a rotation of the second rotatable member. In particular, in response to the rotation of the second rotatable member, the engagement preventing member may be wound around the second rotatable member. The engagement preventing member may be wound around the first rotatable member in the first state of the mechanism and the engagement preventing member may be unwound from the first rotatable member by a rotation of the second rotatable member.

Furthermore, the body of the mechanism may comprise another pin wherein the second rotatable member is configured to be arranged on the other pin and to rotate around the other pin. In particular, the second rotatable member may be configured to rotate around an axis being parallel to the second rotational axis. The axis may run through the centre point of the second rotatable member.

The blocking member may comprise a first tension member and a second tension member. When the blocking feature is in the first position, the first tension member may be tensioned in one direction by the first rotatable member and the second tension member may be tensioned in an opposite direction by the second rotatable member, thereby tensing the blocking member to rotate. During operation of the mechanism, the diameter of the first rotatable member may decrease and the diameter of the second rotatable member may increase. However, the tension of the first and the second tension member is not changing during operation of the mechanism in the first state. In this context, the term “diameter of the first rotatable member” shall refer to the diameter of the first rotatable member including the part of the engagement preventing member wound around the first rotatable member. Respectively, the term “diameter of the second rotatable member” shall refer to the diameter of the second rotatable member including the part of the engagement preventing member wound around the second rotatable member.

The blocking member may be configured to rotate when the mechanism changes from its first state to its second state. In particular, the blocking member may be configured to rotate around a third rotational axis being parallel to the first and the second rotational axis. The third rotational axis may run through a centre point of the blocking member.

Furthermore, the engagement preventing member may comprise a tape comprising indicia configured to display information regarding the drug delivery device. In particular, the tape may be configured to display a number counted by the mechanism. The counted number may correspond to the number of doses left in the device or to the number of dose dispensing operations that have been performed.

According to a second aspect, the present disclosure relates to an assembly for a drug delivery device comprising the above-discussed mechanism and a second mechanism which comprises an interaction member configured to be moved during at least one of a dose dispensing operation and a dose setting operation of the drug delivery device. The mechanism may be the above-discussed mechanism such that every structural and functional feature discussed with respect to this mechanism may also be presenting the assembly.

The second mechanism may be a dose dispensing mechanism and/or a dose setting mechanism.

The blocking member may be configured to prevent a movement of the interaction member in the second state of the mechanism. In particular, the blocking feature of the blocking member may prevent this movement. Thereby, at least one of a dose setting operation and a dose dispensing operation of the drug delivery device may be prevented in the second state of the mechanism. A further operation of the drug delivery device is no longer possible in the second state of the mechanism. Thereby the user is warned and it is prevented that the user carries out a dose dispensing operation without a dose being dispensed and without the user noticing this.

Furthermore, the assembly may comprise a guiding track wherein the interaction member moves along the guiding track during at least one of the dose setting operation and the dose dispensing operation. In its second position, the blocking feature is arranged in the guiding track, thereby blocking the movement of the interaction member along the guiding track. Furthermore, in its first position, the blocking feature may be arranged outside the guiding track such that the interaction member is enabled to move along the guiding track without interference of the blocking feature.

The interaction member may be configured to rotate the second rotatable member when the interaction member engages with the second rotatable member. In particular, the second rotatable member may comprise a helical thread which is configured to be engaged with the interaction member. The helical thread may be shaped such that the interaction member engages with the helical thread at the beginning of a dose setting operation or a dose dispensing operation. Furthermore, the interaction member may be disengaged from the helical thread near the end of the respective dose setting operation or the respective dose dispensing operation. In particular, the interaction member may reach the end of the helical thread and may thereby be disengaged from the helical thread near the end of the respective operation.

According to another aspect, the present disclosure relates to a drug delivery device comprising either the above-discussed mechanism or comprising the above-discussed assembly. Thus, every structural and functional feature disclosed with respect to either the mechanism or the assembly may also be present with respect to the drug delivery device.

The term “medical substance” or “substance”, as used herein may mean a pharmaceutical formulation containing at least one pharmaceutically active compound, for example for the treatment of obstructive airway or lung diseases such as asthma or chronic obstructive pulmonary disease (COPD), local respiratory tract oedema, inflammation, viral, bacterial, mycotic or other infection, allergies, diabetes mellitus.

The active pharmaceutical compound is preferably selected from the group consisting of active pharmaceutical compounds suitable for inhalation, preferably antiallergenic, antihistamine, anti-inflammatory, antitussive agents, bronchodilators, anticholinergic drugs, and combinations thereof.

The active pharmaceutical compound may for example be chosen from:

an insulin such as human insulin, e.g. a recombinant human insulin, or a human insulin analogue or derivative, a glucagon-like peptide (GLP-1) or an analogue or derivative thereof, or exendin-3 or exendin-4 or an analogue or derivative of exendin-3 or exendin-4;
an adrenergic agent such as a short acting β2-agonists (e.g. Salbutamol, Albuterol, Levosalbutamol, Fenoterol, Terbutaline, Pirbuterol, Procaterol, Bitolterol, Rimiterol, Carbuterol, Tulobuterol, Reproterol), a long acting β2-agonist (LABA, e.g. Arformoterol, Bambuterol, Clenbuterol, Formoterol, Salmeterol), an ultra LABA (e.g. Indacaterol) or another adrenergic agent (e.g. Epinephrine, Hexoprenaline, Isoprenaline (Isoproterenol), Orciprenaline (Metaproterenol));
a glucocorticoid (e.g. Beclometasone, Budesonide, Ciclesonide, Fluticasone, Mometasone, Flunisolide, Betamethasone, Triamcinolone);
an anticholinergic agent or muscarinic antagonist (e.g. Ipratropium bromide, Oxitropium bromide, Tiotropium bromide);
a mast cell stabilizer (e.g. Cromoglicate, Nedocromil);
a xanthine derivative (e.g. Doxofylline, Enprofylline, Theobromine, Theophylline, Aminophylline, Choline theophyllinate);
an eicosanoid inhibitor, such as a leukotriene antagonist (e.g. Montelukast, Pranlukast, Zafirlukast), a lipoxygenase inhibitor (e.g. Zileuton) or a thromboxane receptor antagonist (e.g. Ramatroban, Seratrodast);
a phosphodiesterase type-4 inhibitor (e.g. Roflumilast);
an antihistamine (e.g. Loratadine, Desloratadine, Cetirizen, Levocetirizine, Fexofenadine);
an allergen immunotherapy (e.g. Omalizumab);
a mucolytic (e.g. Carbocisteine, Erdosteine, Mecysteine);
an antibiotic or antimycotic;
or a combination of any two, three or more of the above-mentioned compound classes or compounds (e.g. Budesonide/Formoterol, Fluticasone/Salmeterol, Ipratropium bromide/Salbutamol, Mometasone/Formoterol);
or a pharmaceutically acceptable salt or solvate or esters of any of the above named compounds.

Pharmaceutically acceptable salts are for example acid addition salts and basic salts. Acid addition salts are e.g. a chloride, bromide, iodide, nitrate, carbonate, sulfate, methylsulfate, phosphate, acetate, benzoate, benzenesulfonate, fumarate, malonate, tartrate, succinate, citrate, lactate, gluconate, glutamate, edetate, mesylate, pamoate, pantothenate or a hydroxy-naphthoate salt. Basic salts are for example salts having a cation selected from alkali or alkaline, e.g. Na+, or K+, or Ca2+, or an ammonium ion N+(R1)(R2)(R3)(R4), wherein R1 to R4 independently of each other mean: hydrogen, an optionally substituted C1-C6-alkyl group, an optionally substituted C2-C6-alkenyl group, an optionally substituted C6-C10-aryl group, or an optionally substituted C6-C10-heteroaryl group. Further examples of pharmaceutically acceptable salts are described in “Remington's Pharmaceutical Sciences” 17. ed. Alfonso R. Gennaro (Ed.), Mark Publishing Company, Easton, Pa., U.S.A., 1985 and in Encyclopedia of Pharmaceutical Technology. Pharmaceutically acceptable ester may for example be acetates, propionates, phosphates, succinates or etabonates.

Pharmaceutically acceptable solvates are for example hydrates.

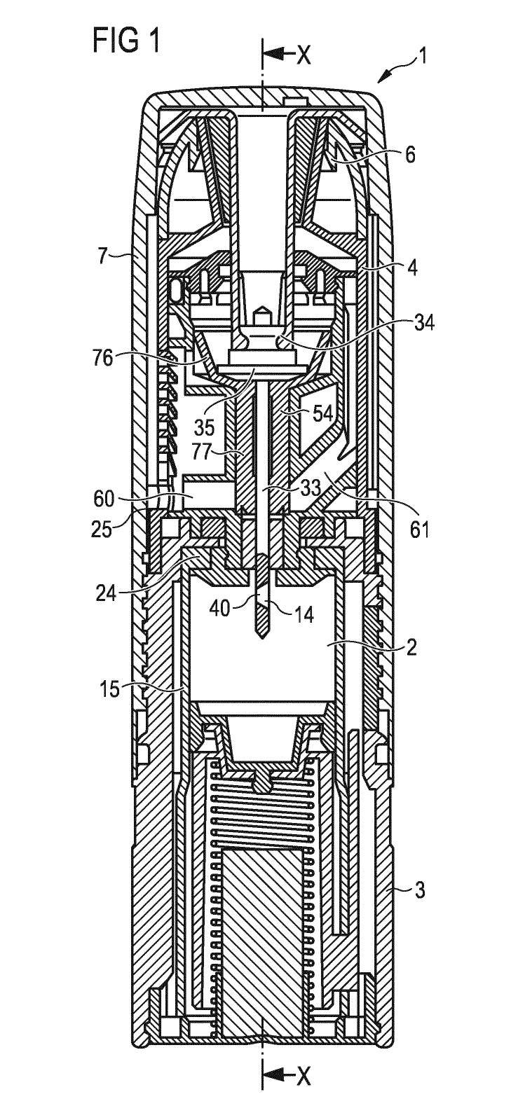

FIG. 1 shows a sectional side view of an inhalation device.

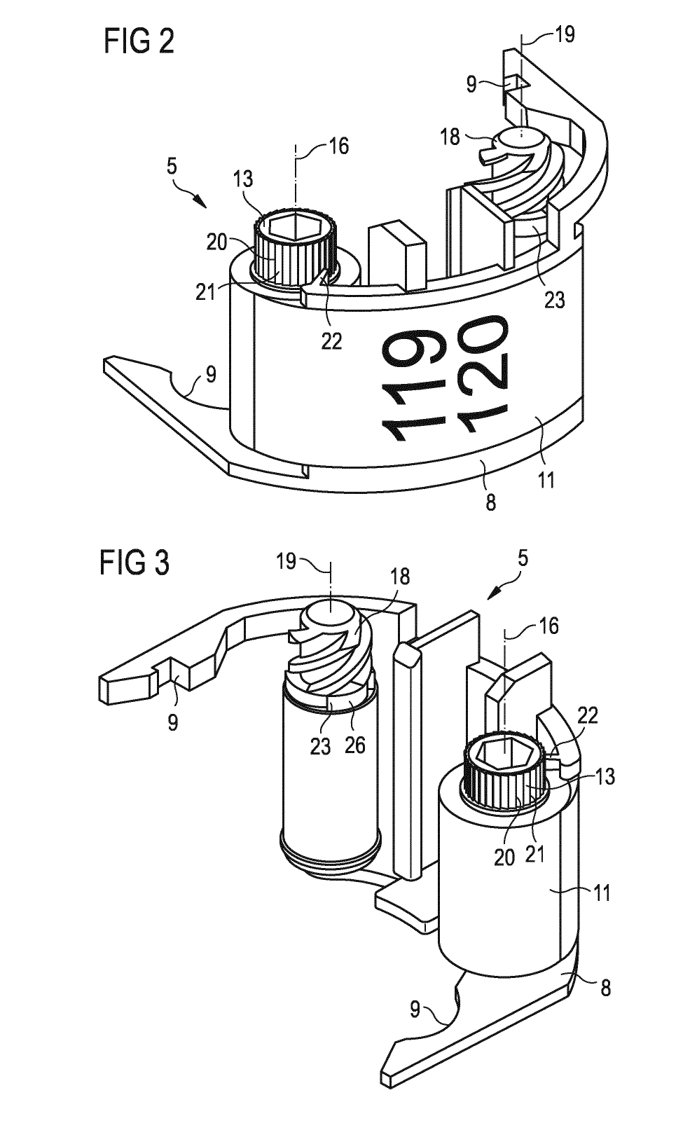

FIGS. 2 and 3 show perspective views of a mechanism according to a first embodiment.

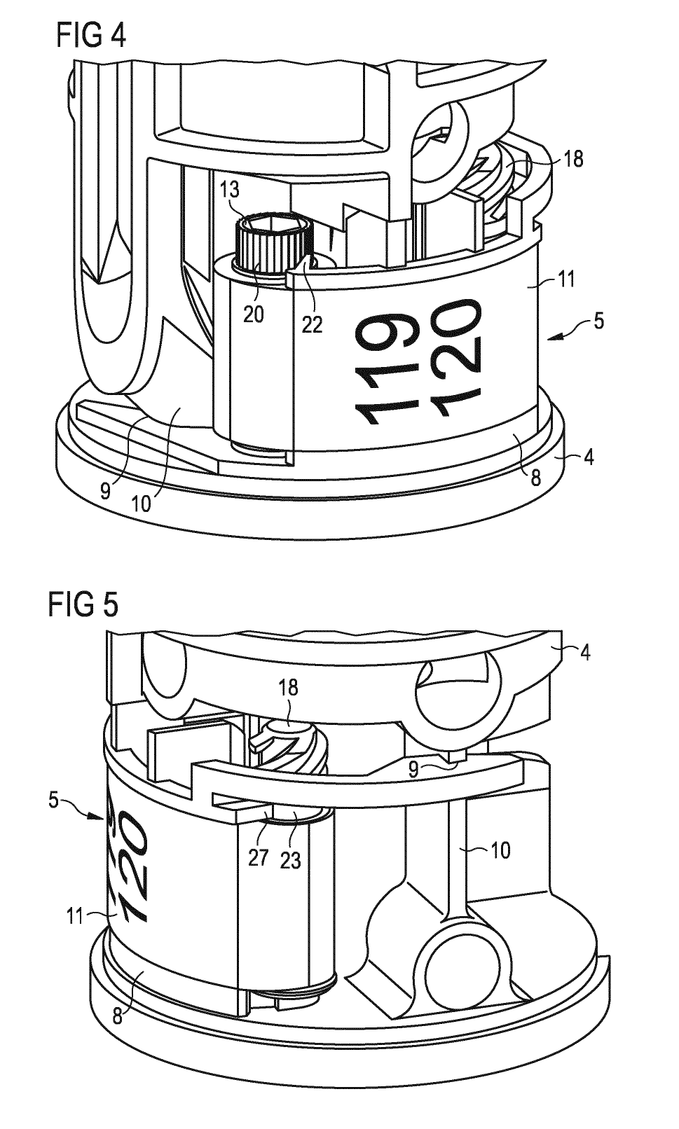

FIGS. 4 and 5 show perspective views of the inhalation device comprising the mechanism.

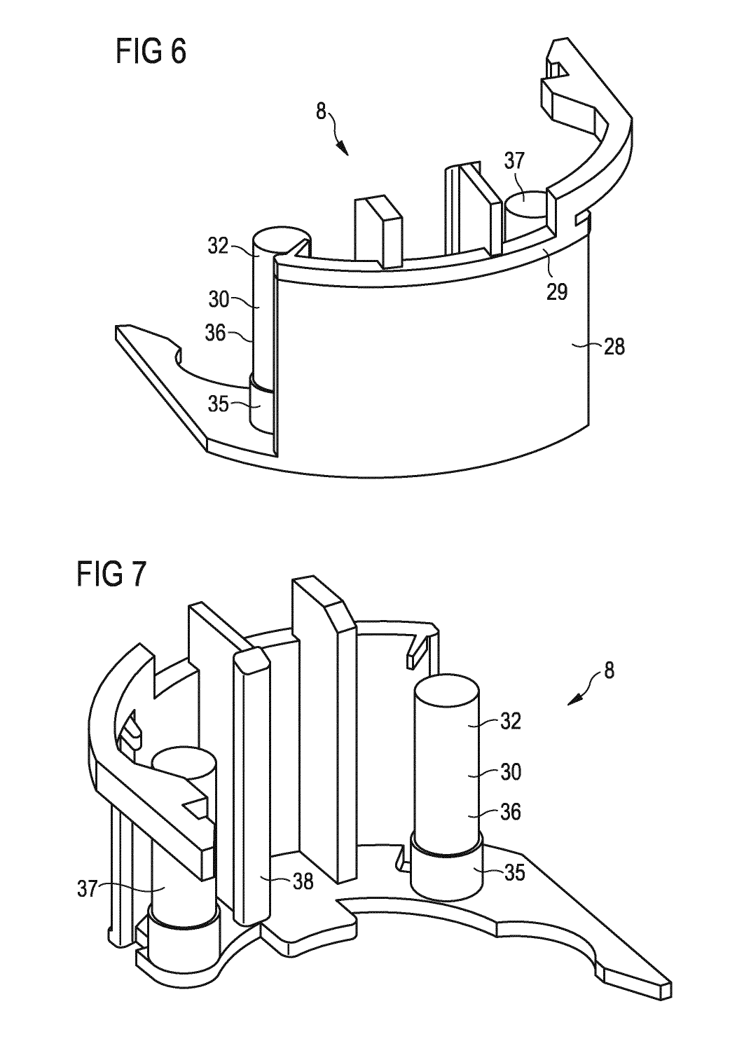

FIGS. 6 and 7 show perspective views of a body of the mechanism.

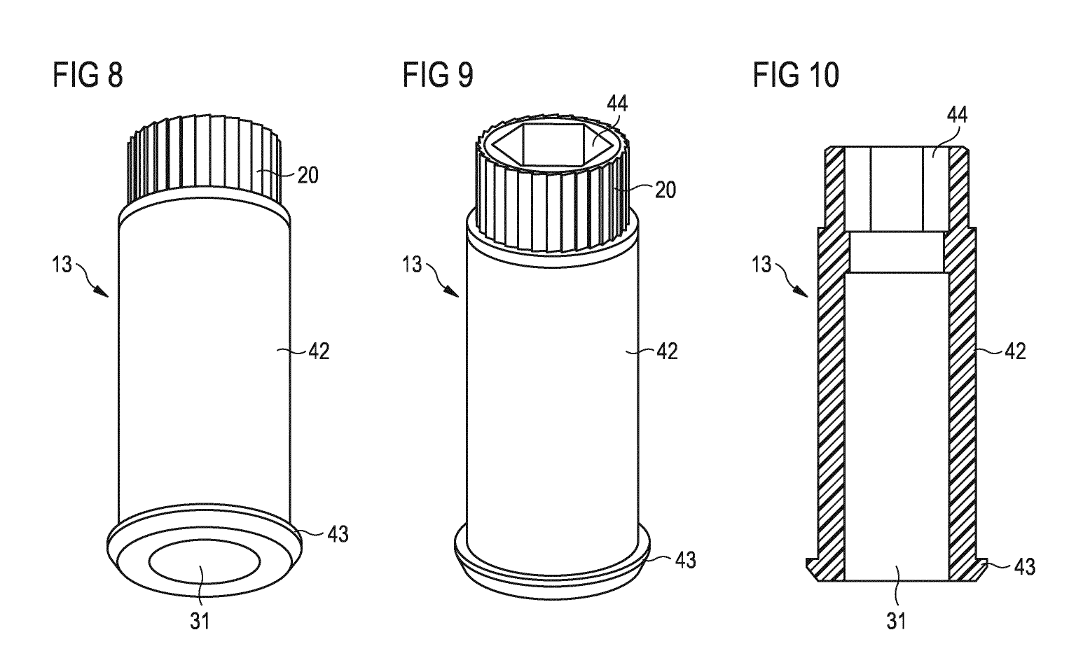

FIGS. 8 and 9 show perspective views of a first rotatable member.

FIG. 10 shows a cross-sectional view of the first rotatable member.

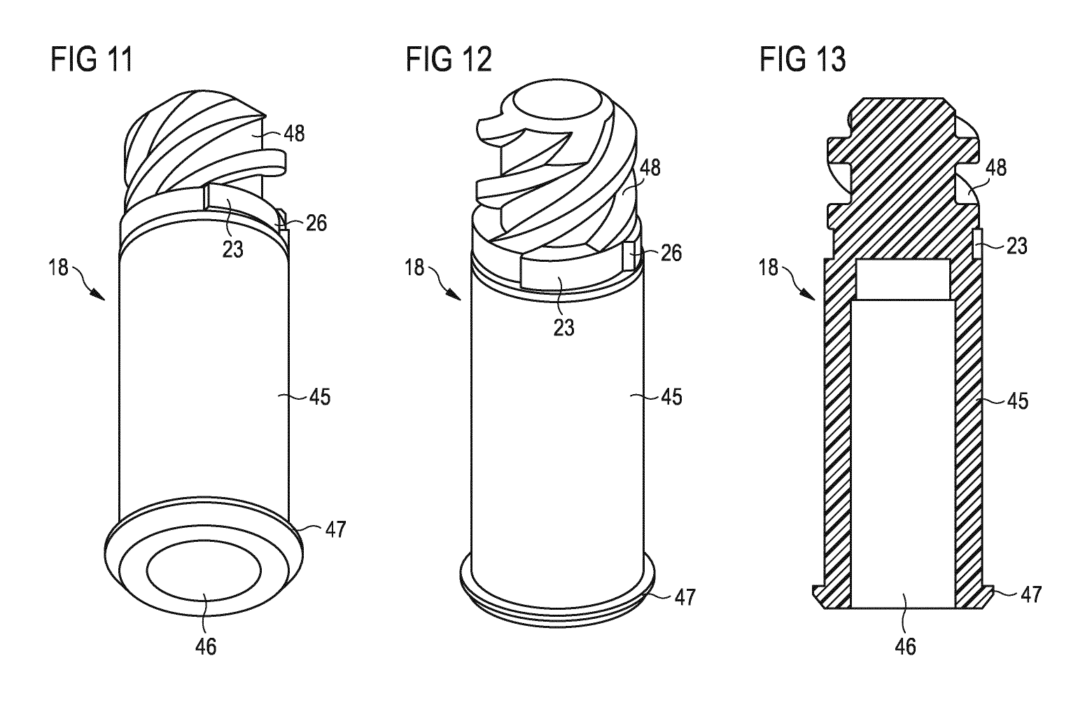

FIGS. 11 and 12 show perspective views of a second rotatable member.

FIG. 13 shows a cross-sectional view of the second rotatable member.

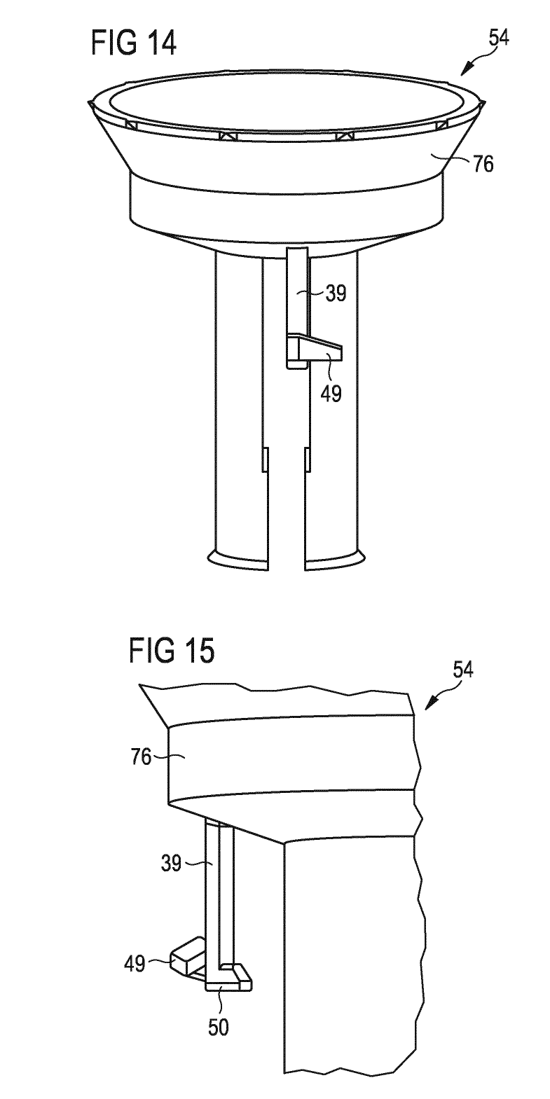

FIG. 14 shows a perspective view of an actuating element.

FIG. 15 shows a more detailed view of a part of the actuating element.

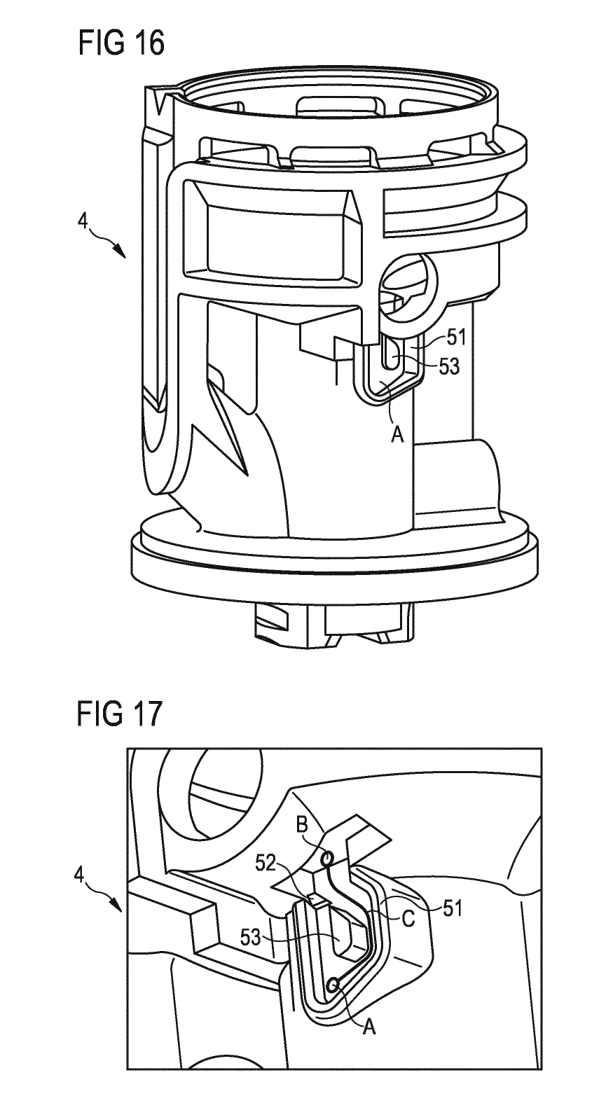

FIG. 16 shows a perspective of an outer cylinder of the inhalation device.

FIG. 17 shows a more detailed perspective view of the outer cylinder.

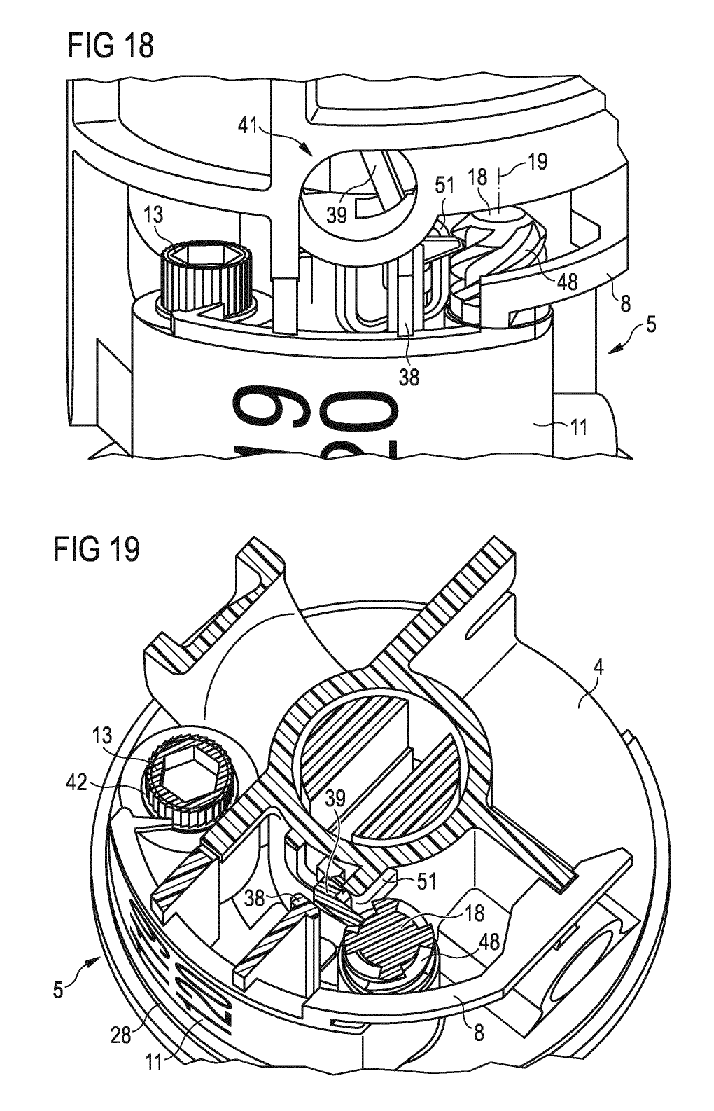

FIGS. 18 and 19 show perspective views of the engagement of an interaction member with the second rotatable member.

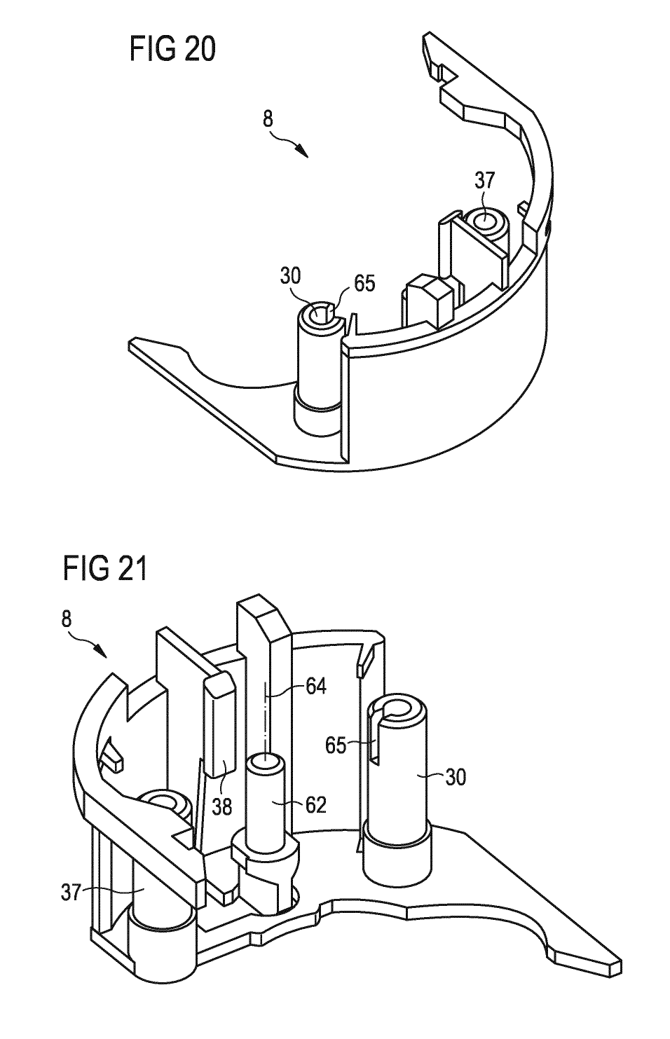

FIGS. 20 and 21 show perspective views the body of the mechanism according to a second embodiment.

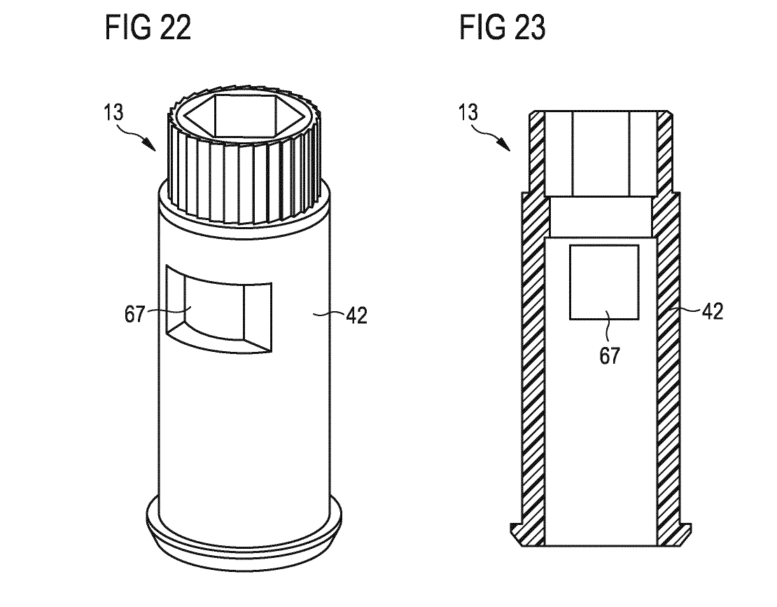

FIG. 22 shows a perspective view of the first rotatable member according to the second embodiment.

FIG. 23 shows a cross-sectional view of the first rotatable member according to the second embodiment.

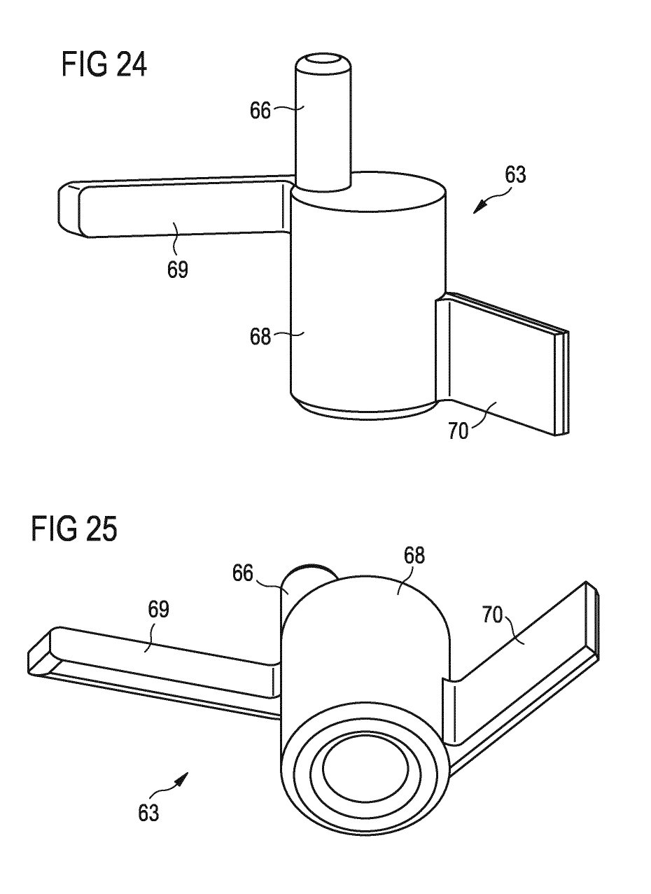

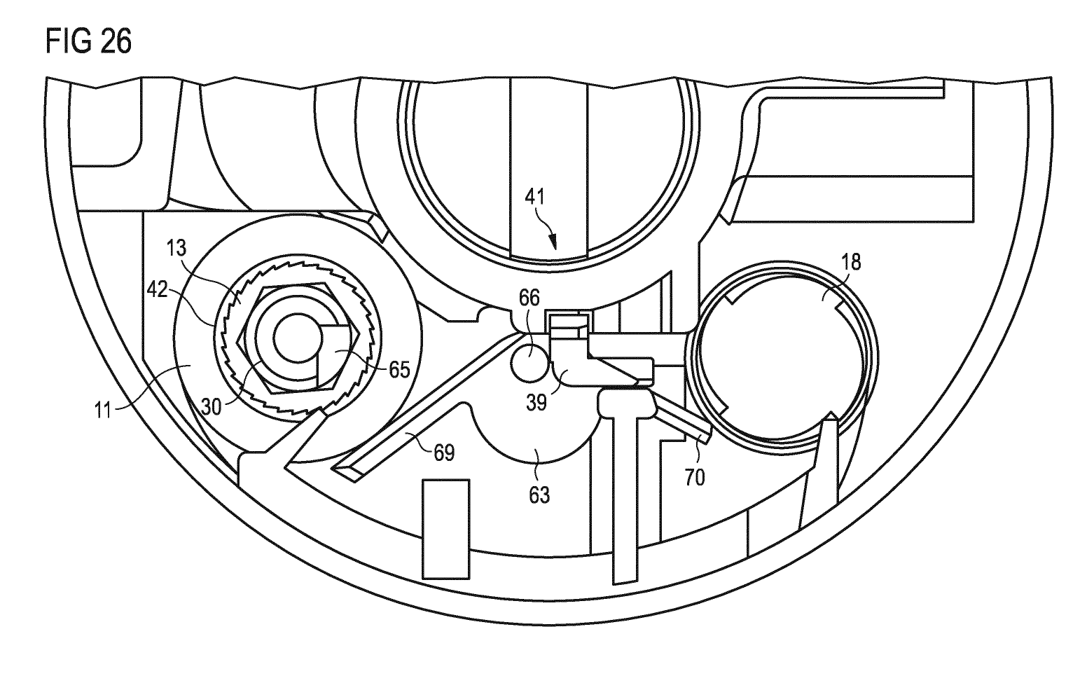

FIGS. 24 and 25 show perspective views of a blocking member.

FIG. 26 shows a cross-sectional view of the inhalation device comprising the mechanism according to the second embodiment in the first state of the mechanism.

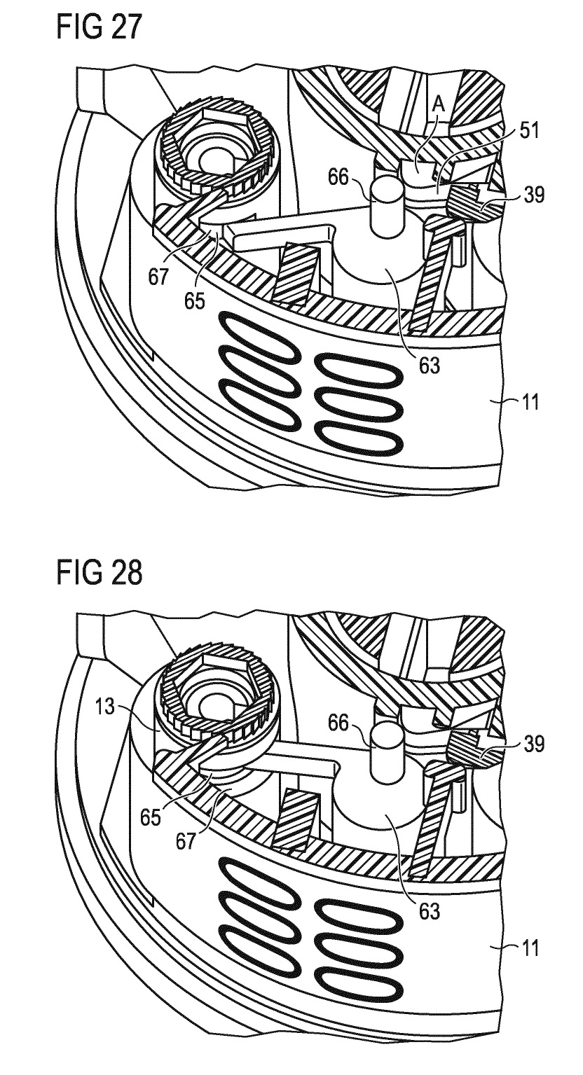

FIG. 27 shows a perspective view of the inhalation device comprising the mechanism according to the second embodiment after the last dose has been delivered when the mechanism changes from its first state to its second state.

FIG. 28 shows the mechanism in its second state.

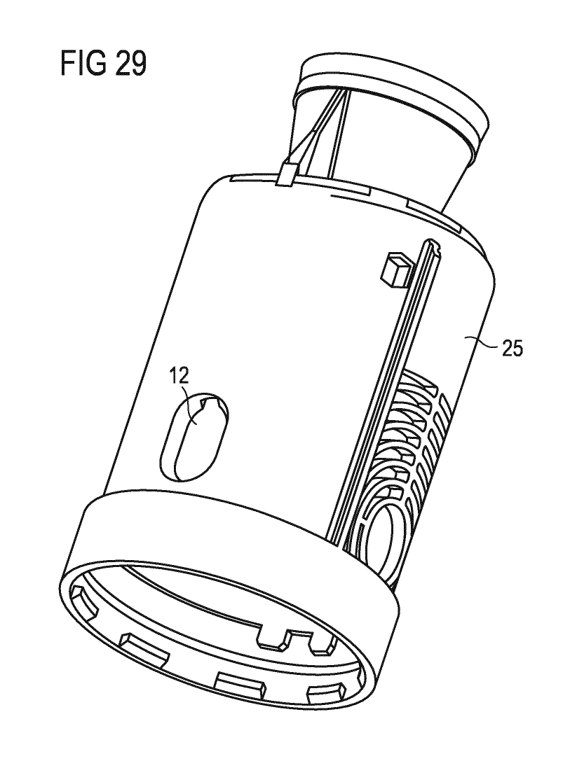

FIG. 29 shows a perspective view of a rotary body of the inhalation device.

Like elements, elements of the same kind and identically acting elements may be provided with the same reference numerals in the figures.

In FIG. 1, a sectional side view of an inhalation device 1 is shown.

The inhalation device 1 comprises a housing 3. The device 1 comprises an outer cylinder 4. The outer cylinder 4 is secured against axial movement with respect to the housing 3. The outer cylinder 4 is rotatable with respect to the housing 3.

The inhalation device 1 further comprises a mouthpiece 6. The inhalation device 1 comprises a cap 7. The cap 7 is used for covering the mouthpiece 6. The cap 7 may comprise a thread, preferably a screw thread. The cap 7 may be rotatable with respect to the housing 3 for screwing the cap 7 onto the device 1 and for unscrewing the cap 7 from the device 1. The outer cylinder 4 is rotationally fixed to the cap 7. In particular, the outer cylinder 4 follows rotation of the cap 7 with respect to the housing 3. For the detailed description of the components of the inhalation device 1 and their mechanical cooperation it is referred to document WO 2009/065707 A1.

The device 1 comprises a storage chamber 15. The storage chamber 15 holds one dose, preferably a plurality of doses, of a medical substance 2. The substance 2 may be a powder.

In particular, the plurality of doses may correspond to a predefined number of doses, such that after the predefined number of doses has been delivered a lock-out mechanism may prevent a further operation of the device. The lock-out mechanism is not shown in FIG. 1, but will be discussed in detail later on.

A numerical value corresponding to the predefined number of doses is a starting value of a dose counting mechanism. Before the first dose delivery, the dose counting mechanism displays this predefined number as the number of available doses and with every dose delivery the number is decremented. Alternatively, the dose counting mechanism may display the number of doses that already have been delivered. In this case, the dose counting mechanism displays “0” as a predefined number before the first dose delivery and with every dose delivery the number is incremented. The dose counting mechanism is not shown in FIG. 1, but the dose counting mechanism will be discussed in detail later on.

The storage chamber 15 is terminated by a chamber ceiling 24. The chamber sealing 24 is formed integrally with a top wall of the storage chamber 15. The device 1 further comprises a rotary part 25. The rotary part 25 is of substantially plate-like configuration and is connected in a rotationally fixed manner to the outer cylinder 4. Accordingly, the rotary part 25 follows rotation of the cap 7 and, hence, of the outer cylinder 4 about a main longitudinal axis x of the device 1 with respect to the storage chamber 15. However, the rotary body 25 is axially fixed relative to the housing 3.

The device 1 further comprises a metering rod 33. The metering rod 33 may be connected to the cap 7 by a snap fit element 34 when the cap 7 is engaged to the housing 3. When the metering rod 33 is connected to cap 7 by the snap fit element 34 a rotation of the metering rod 33 relative to the cap 7, and thereby also relative to the rotary part 25, is prevented. Accordingly, the metering rod 33 follows rotational movement of the cap 7 and, hence, of the rotary part 25 about the main longitudinal axis x when the cap 7 is mounted onto the device 1 or demounted from the device 1.

When the cap 7 is re-engaged to the housing 3 the metering rod 33 travels axially in the proximal direction such that the most proximal part of the metering rod 33 comprising a metering chamber 40 enters the storage chamber 15. When the cap 7 is disengaged from the housing 3 the metering rod 33 travels axially in the distal direction such that the most proximal part of the metering rod 33 exits the storage chamber 15. In this context, “distal” may refer to the end of the inhalation device closest to the mouthpiece 6. Accordingly, “proximal” may refer to the end of the inhalation device furthest away from the mouthpiece 6.

In particular, the metering rod 33 is configured for functioning as a moving metering chamber 40 for a sub-quantity 14 of the substance 2 which is to be dispensed during a specific delivery action. The metering chamber 40 is formed in that end section of the metering rod 33 which projects into the substance 2.

The inhalation device 1 further comprises a flow path comprising a flow channel 60 and an intermediate channel portion 61.

The inhalation device 1 further comprises an actuating element 54. The actuating element 54 comprises a piston comprising tongues 77 and a head 76. The actuating element 54 has a first and a second position. The first position is more proximal than the second position. In the first position, the tongues 77 of the actuating element 54 block the flow path between the flow channel 60 and the intermediate channel portion 61. In its second position, the actuating element 54 is positioned more distally such that the tongues 77 do not block the flow path between the flow channel 60 and the intermediate channel portion 61 anymore.

The cap 7 is removed from the housing 3 by unscrewing the cap 7 from the housing 3. Accordingly, the cap 7 performs concurrently an axial movement in the distal direction and a rotational movement. The cap 7 and the rotary part 25 are in a splined engagement when the cap is attached to the housing 3. During disengagement of the cap 7 from the housing 3, the rotational movement of the cap 7 is transferred into a rotation of the rotary part 25 around the longitudinal axis x due to their splined engagement. The rotation of the rotary part 25 is transferred into a rotation of the actuating element 54. Furthermore, the concurrent axial and rotational movement of the cap 7 is transferred to the metering rod 33 concurrently performing an axial movement in the distal direction and a rotational movement around the longitudinal axis x. As the cap 7 approaches the end of the threaded connection to the housing 3, the snap fit element 34 is disengaged from the metering rod 33.

During disengagement of the cap 7 from the housing 3, the actuating element 54 is not moved axially relative to the housing 3. Accordingly, the actuating element 54 is in its first position before and after disengagement of the cap 7 from the housing 3.

When the cap 7 is fully disengaged from the housing 3, the metering chamber 40 is in a first condition. The first condition of the metering chamber 40 is defined by the tongues 77 of the actuating element 54 closing the metering chamber 40 such that the metering chamber 40 is not in contact with the flow path. Accordingly, when the actuating element 54 is in its first position and the cap 7 is disengaged from the housing 3, the metering chamber 40 is in its first condition.

In the first condition of the metering chamber 40, the tongues 77 of the actuating element 54 cover the metering chamber 40 on each side. Accordingly, in this first condition, it is not possible for the sub-quantity 14 of substance to trickle out. Rather, the substance is reliably retained in the metering chamber 40.

After the cap 7 has been demounted, the user may trigger an inhalation operation by subjecting the device to a suction airstream, in the simplest case by the user breathing in. Air is sucked in via the mouthpiece 6, and this, in first instance, by virtue of the head 76 being subjected to the action of air, results in the actuating element 54 being displaced axially in the direction of the mouthpiece 6.

By virtue of the axially displaced actuating element 54, the tongues 77 are likewise displaced axially, in order to release the metering chamber 40. The metering chamber 40 is then in a second condition. The second condition of the metering chamber 40 is defined by the actuating element 54 being in its second position. In its second condition, the metering chamber 40 lies freely in a flow path between a flow channel 60 and an intermediate channel portion 61. The metering chamber is cleared out with air being sucked from the flow channel 60.

After the inhalation operation, the cap 7 may be engaged to the housing 3. During engagement of the cap 7 to the housing 3, the cap 7 is moved axially in the proximal direction and concurrently rotated around the longitudinal axis x. The snap fit element 34 engages to the metering rod 33 at the beginning of the threaded connection. Thereby the metering rod 33 is rotated and moved into the proximal direction when the cap 7 is engaged to the housing 3.

During reengagement of the cap 7 to the housing 3, the metering rod 33 is moved in a proximal direction due to the interaction with the cap 7. If the actuating element 54 is in its second position and the metering rod 33 is moved in the proximal direction, this movement is transferred to the actuating element 54. Thereby, the actuating element 54 is moved from its second position to its first position.

However, in case the cap 7 is disengaged from the housing 3 and afterwards reengaged to the housing 3 without a drug delivery being performed in between, the actuating element remains in its first position the whole time. Accordingly, as the actuating element 54 is already in its first position, it can not be moved axially during engagement of the cap 7 to the housing 3.

The inhalation device 1 further comprises a mechanism 5 which, for clarity reasons, is not depicted in FIG. 1. According to a first embodiment, the mechanism 5 counts the number of doses left in the inhalation device 1. The mechanism 5 is further configured to display the counted number corresponding e.g. to the number of doses left in the device 1. Alternatively, the counted number may correspond to another quantity, e.g. the number of doses which have been delivered by the inhalation device 1. Moreover, according to a second embodiment, the mechanism 5 is additionally configured to prevent a further operation of the device 1 after a certain number of doses has been delivered. Thus, the mechanism 5 according to the second embodiment is a combined dose counting and lock-out mechanism. In another alternate embodiment, the mechanism 5 may be a lock-out mechanism which does not display the counted number.

FIGS. 2 and 3 show perspective views of the mechanism 5 according to the first embodiment. FIGS. 4 and 5 show perspective views of the inhalation device 1 comprising the mechanism 5.

As will be discussed in the following, the mechanism 5 has a first state and a second state.

The mechanism 5 comprises a body 8. The body 8 is configured to be snap-fitted to the outer cylinder 4 of the inhalation device 1. For this purpose, the body 8 and the outer cylinder 4 comprise corresponding snap-fit elements 9, 10. In particular, the body 8 comprises a first and a second arm, each comprising a snap-fit element 9 that may be engaged with corresponding snap-fit elements 10 of the outer cylinder 4, e.g. with corresponding protrusions, thereby fixing the body 8 of the mechanism 5 to the outer cylinder 4.

Furthermore, the mechanism 5 comprises an engagement preventing member 11. According to the first embodiment, the engagement preventing member 11 is a counting member configured to count and to display the counted number. The engagement preventing member 11 comprises a tape.

The engagement preventing member 11 comprises numbers arranged on the engagement preventing member 11. In particular, the numbers are arranged on the engagement preventing member 11 such that one number is visible in a window 12 [not shown in FIGS. 2 and 3] of the rotary body 25 in each position of the engagement preventing member 11. This number corresponds to the counted number of the mechanism 5.

Moreover, the mechanism 5 comprises a first rotatable member 13. The first rotatable member 13 comprises a pulled wheel which is configured to rotate around a second rotational axis 16. One end of the engagement preventing member 11 is attached to the first rotatable member 13. The one end may be fixed to the first rotatable member 13 or may be releasably attached to the first rotatable member 13. In this context, the term “one end being fixed to the first rotatable member 13” means that the one end can only be removed from the first rotatable member 13 by damaging the engagement preventing member 11.

In the first state of the mechanism 5, the engagement preventing member 11 is wound around the first rotatable member 13 such that the engagement preventing member 11 covers a first surface 42 [not shown in FIGS. 2 and 3] of the first rotatable member 11. The surface normal of the first surface 42 is perpendicular to the second rotational axis 16.

Furthermore, the mechanism 5 comprises a second rotatable member 18. The second rotatable member 18 comprises a pulling wheel. A second end of the engagement preventing member 11 is attached to the second rotatable member 18. The second end is attached either by a fixed attachment or by a releasable attachment to the second rotatable member 18. The second rotatable member 18 is configured to rotate around a third rotational axis 19. The third rotational axis 19 is parallel to the second rotational axis 16.

The engagement preventing member 11 is mounted to the first and to the second rotatable member 13, 18 such that, in response to a rotation of the second rotatable member 18 in a first rotational direction, the engagement preventing member 11 is wound around the second rotatable member 18. Simultaneously, in response to a rotation of the second rotatable member 18 in the first rotational direction, the engagement preventing member 11 is unwound from the first rotatable member 13. As the engagement preventing member 11 is unwound from the first rotatable member 13, the first rotatable member 13 is rotated in the first rotational direction.

When the engagement preventing member 11 is unwound from the first rotatable member 13 the diameter of the first rotatable member 13 decreases. When the engagement preventing member 11 is wound on the second rotatable member 18, the diameter of the second rotatable member 18 increases. In this context, the diameter of the first rotatable member 13 is measured in a direction perpendicular to the second rotational axis 16. Furthermore, the term “diameter of the first rotatable member 13” shall refer to the diameter of the first rotatable member 13 including the part of the engagement preventing member 11 wound around the first rotatable member 13. Respectively, the diameter of the second rotatable member 18 is measured in a direction perpendicular to the third rotational axis 19. The term “diameter of the second rotatable member 18” shall refer to the diameter of the second rotatable member 18 including the part of the engagement preventing member 11 wound around the second rotatable member 18.

The first rotatable member 13 comprises a first reverse rotation prevention feature 20 which is configured to permit a rotation of the first rotatable member 13 in the first rotational direction and which is further configured to prevent a rotation of the first rotatable member 13 in a second rotational direction which is opposite to the first rotational direction. In particular, the first reverse rotation prevention feature 20 comprises teeth 21 arranged on the first rotatable member 18 which are configured to cooperate with a first pawl 22 of the body 8. The first reverse rotation prevention feature 20 and the first pawl 22 are shaped such that a rotation of the first rotatable member 18 in the second rotational direction is prevented. Furthermore, the first reverse rotation prevention feature 20 and the first pawl 22 are shaped such that a rotation of the first rotatable member 13 in the first rotational direction is allowed.

Moreover, the second rotatable member 18 comprises a second reverse rotation prevention feature 23 which is configured to permit a rotation of the second rotatable member 18 in the first rotational direction and which is further configured to prevent a rotation of the second rotatable member 18 in the second rotational direction. The second reverse rotation prevention feature 23 is constructed in the same way as the first reverse rotation prevention feature 20. In particular, the second reverse rotation prevention feature 23 comprises teeth 26 arranged on the second rotatable member 18 which are configured to cooperate with a second pawl 27 [not shown in FIGS. 2 and 3] of the body 8. The second reverse rotation prevention feature 23 and the second pawl 27 are shaped such that a rotation of the second rotatable member 18 in the second rotational direction is prevented. Furthermore, the second reverse rotation prevention feature 23 and the second pawl 27 are shaped such that a rotation of the second rotatable member 18 in the first rotational direction is allowed.

The first and the second reverse rotation prevention feature 20, 23 differ in the number of teeth 21, 26. The second reverse rotation prevention feature 23 comprises four teeth 26 corresponding to the second rotatable member 18 being rotated by a quarter of a complete rotation during each operation of the mechanism 5, as will be discussed later on. The first reverse rotation prevention feature 20 comprises multiple teeth 21 allowing the first reverse rotation prevention feature 20 to be engaged with the first pawl 22 in multiple positions.

In FIGS. 4 and 5, the engagement of the first and the second pawl 22, 27 of the body 8 with the first and the second reverse rotation prevention feature 20, 23 is shown. The first and the second pawl 22, 27 also ensure that the engagement preventing member 11 is held in the correct position. In particular, the pawls 22, 27 prevent the engagement preventing member 11 from moving relative to the first rotatable member 13 or, respectively, the second rotatable member 18 in the distal direction.

FIGS. 6 and 7 show perspective views of the body 8 of the mechanism 5. The body 8 comprises a guidance face 28 configured to guide the engagement preventing member 11 when said engagement preventing member 11 is unwound from the first rotatable member 13 and wound around the second rotatable member 18. The guidance face 28 comprises a protruding flange 29 preventing the engagement preventing member 11 from moving in the distal direction.

Moreover, the body 8 comprises a first pin 30 which is configured such that the first rotatable member 11 may be arranged on the first pin 30. In particular, the first rotatable member 11 comprises an opening 31 [not shown in FIGS. 6 and 7] configured to receive the first pin 30. The first rotatable member 11 is configured to rotate around the first pin 30. The first pin 30 has a first end portion 32, a second end portion 35 and a middle portion 36 arranged between the first and the second end portions 32, 35. When the mechanism 5 is attached to the inhalation device 1, the first end portion 32 is arranged at a distal end of the first pin 30 and the second end portion 35 is arranged at a proximal end of the first pin 30. The diameter of the first pin 30 in the middle portion 36 is smaller than the diameter of the first pin 30 in the first and the second end portion 32, 35.

Moreover, the body 8 comprises a second pin 37 which is constructed in the same way as the first pin 30.

Furthermore, the body 8 comprises a guidance element 38. The guidance element 38 is an elongated element configured to extend in a direction parallel to the longitudinal axis x of the inhalation device 1 when the mechanism 5 is attached to the inhalation device 1. The guidance element 38 is configured to constrain a movement of an interaction member 39 [not shown in FIGS. 6 and 7] of a second mechanism 41 [not shown in FIGS. 6 and 7] which will be discussed later on. Thereby, the guidance element 38 is configured to guide the movement of the interaction member 39.

FIGS. 8 and 9 show perspective views of the first rotatable member 13. FIG. 10 shows a cross-sectional view of the first rotatable member 13.

The first rotatable member 13 comprises the first surface 42. The engagement preventing member 11 may be wound around the first surface 42. The engagement preventing member 11 is configured to cover the first surface 42 at least partially. In the first state of the mechanism 5, the engagement preventing member 11 covers the first surface 42 at least partly. In the second state of the mechanism 5, the engagement preventing member 11 covers a smaller part of the first surface 42 than in the first state of the mechanism 5.

The first reverse rotation prevention feature 20 of the first rotatable member 13 is arranged adjacent to the first surface 42. In particular, when the mechanism 5 is attached to the inhalation device 1, the first reverse rotation prevention feature 20 is arranged distally of the first surface 42. On the side of the first surface 42 opposite to the first reverse rotation prevention feature 20, the first rotatable member 13 comprises an edge 43. In particular, the diameter of the first rotatable member 13 is increased to form the edge 43. The edge 43 prevents a movement of the engagement preventing member 11 in a direction away from the first reverse rotation prevention feature 20. Thus, the engagement preventing member 11 is prevented from moving in the direction along the second rotatable member 18, as the first pawl 22 prevents a movement of the engagement preventing member 11 in a direction towards the first reverse rotation prevention feature 20 and as the edge 43 prevents a movement of the engagement preventing member 11 in a direction away from the first reverse rotation prevention feature 20.

The diameter of the first rotatable member 13 reduces starting from a maximal diameter at the edge 43 in a direction away from the first reverse rotation prevention feature 20. Thereby, the surface of the first rotatable member 13 which is in contact with the body 8 when the first rotatable member 13 is arranged on the first pin 30 is reduced.

As can be seen in FIG. 10, the first rotatable member 13 comprises the opening 31 configured to receive the first pin 30. The diameter of the opening 31 and the diameter of the sections 32, 36, 37 of the first pin 30 are chosen such that the first rotatable member 13 contacts the first pin 30 only in the first and the second end portion 32, 35. Thus, the first rotatable member 13 is configured to rotate on the first pin 30 with a very low friction.

Furthermore, the first rotatable member 30 comprises a tool receiving opening 44 configured to receive a tool. The tool may be configured to rotate the first rotatable member 13. In particular, it is possible to exert a force via the tool which rotates the first rotatable member 13. If the first and the second reverse rotation prevention feature 20, 23 are disabled, the tool can be used to rotate the first rotatable member 13 in the second rotational direction.

The opening 31 configured to receive the first pin 30 and the tool receiving opening 44 are connected and form a passage through the first rotatable member 13.

FIGS. 11 and 12 show a perspective view of the second rotatable member 18. FIG. 13 shows a cross-sectional view of the second rotatable member 18. The second rotatable member 18 comprises a first surface 45 constructed in the same way as the first surface 42 of the first rotatable member 13. The first surface 45 is configured such that the engagement preventing member 11 may be wound up on the first surface 45.

Furthermore, the second rotatable member 18 also comprises an opening 46 in its interior configured to receive the second pin 37 wherein the opening 46 has a diameter chosen such that the second rotatable member 18 contacts the second pin 37 only in a first and in a second end portion.

Furthermore, the second rotatable member 18 comprises an edge 47. The edge 47 and the second pawl 27 prevent the engagement preventing member 11 from moving in a direction along the third rotational axis 19, in the same way as described with respect to the edge 43 of the first rotatable member 13 and the first pawl 22.

Moreover, the second rotatable member 18 comprises a helical thread 48. The helical thread 48 is configured such that, when an element, e.g. the interaction member 39, engages the helical thread 48, the second rotatable member 18 is rotated. In particular, the helical thread 48 is a four-start thread. In particular, the helical thread 48 is configured such that the second rotatable member 18 is rotated by roughly 90° when the interaction member 11 engages with the helical thread 48.

More particular, the interaction member 11 may engage one of the four-starts of the helical thread 48 at a distal end. Then, the interaction member 11 slides along the helical thread 48, thereby rotating the second rotatable member 18 by roughly 90°. When the interaction member 11 reaches the proximal end of the helical thread 48, the interaction member 11 is disengaged from the second rotatable member 18.

In contrast to the first reverse rotation prevention feature 20 of the first rotatable member 13, the second reverse rotation prevention feature 23 of the second rotatable member 18 comprises only four teeth 26 corresponding to the number of starts of the helical thread 48. Each tooth 26 is configured to prevent a rotation of the second rotatable member 18 in the second rotational direction.

The mechanism 5 is configured to cooperate with a second mechanism 41 of the inhalation device 1. The second mechanism 41 is a dosing mechanism which allows for a dose setting and/or a dose dispensing operation of the inhalation device 1. The second mechanism 41 comprises the actuating element 54.

FIG. 14 shows a perspective view of the actuating element 54. Furthermore, FIG. 15 shows a more detailed view of a part of the actuating element 54 shown from a different perspective.

The actuating element 54 comprises the head 76 and the interaction member 39. A first end of the interaction member 39 is attached to the head 76. The interaction member 11 comprises a plunger. The interaction member 11 has a first interaction feature 49 and a second interaction feature 50. The first and the second interaction feature 49, 50 are each arranged at an end of the interaction member 39 facing away from the head 76 of the actuating element 54. The first interaction feature 49 comprises a first chamfer extending towards the second rotatable member 18 when the mechanism 5 is attached to the inhalation device 1. The first interaction feature 49 is configured to be engaged with the helical thread 48. The second interaction feature 50 comprises a second chamfer being arranged perpendicular to the first chamfer. The second interaction feature 50 is configured to slide along a guiding track 51 [not shown in FIGS. 14 and 15] of the outer cylinder 4 as will be discussed later on.

The head 76 of the actuating element 54 is formed from a soft material which can be deformed easily. The interaction member 39 is formed from a material which is harder than the soft material of the head 76. Thus, the interaction member 39 is moveable relative to the head 76 of the actuating element 54 in various directions. The interaction member 39 and the head 76 are formed in a two-component molding process. The soft material of the head 76 is molded around the first end of the interaction member 39.

FIG. 16 shows a perspective of the outer cylinder 4 of the inhalation device 1. FIG. 17 shows a more detailed perspective view of the outer cylinder 4 shown in FIG. 16, shown from a different perspective.

The outer cylinder 4 comprises the guiding track 51. When the actuating element 54 is moved from its first to its second position, the interaction member 39 moves along the guiding track 51. When the actuating element 54 is in its first position, the second interaction feature 50 of the interaction member 39 is in a first position A of the guiding track 51. When the actuating element 54 travels from its first to its second position in response to a user's suction airstream, the second interaction feature 50 travels from the first position A to a second position B. The guiding track 51 comprises a ramp 52 which moves the interaction member 39 outwards in a direction away from the longitudinal axis x during the last part of the travel from the first position A to the second position B. Hereby, the second interaction feature 50 slides along the ramp 52.

Furthermore, when the actuating element 54 is moved from its second position B back to its first position A in response to the cap 7 being attached to the inhalation device 1, the interaction member 39 follows this movement. Thereby, the second interaction feature 50 of interaction member 39 travels from the second position B along track C to the first position A. The ramp 52 and a further guide element 53 of the guiding track 51 ensure that the interaction member 39 travels along the track C. Thereby, the first interaction feature 49 of the interaction member 39 is engaged with the helical thread 48 of the second rotatable member 18 when the actuating element 54 is moved from its second position to its first position. The first interaction feature 49 thereby rotates the second rotatable member 18.

Accordingly, when the actuating element 54 is moved from its first position to its second position and back to its first position, the interaction member 39 follows this movement such that the second interaction feature 50 is moved along the guiding track 51. Thereby, the interaction member 39 is engaged with the second rotatable member 18 such that the second rotatable member 18 is rotated.

FIG. 18 and FIG. 19 show perspective views of the engagement of the interaction member 39 with the second rotatable member 18.

When the actuating element 54 is moved from its second position to its first position, the interaction member 39 engages with the helical thread 48 of the second rotatable member 18. Thereby, the second rotatable member 18 is rotated around the third rotational axis 19 by a rotational angle of roughly 90°. Thus, the engagement preventing member 11 is moved one quarter of the circumference of the second rotatable member 18 and wound around the second rotatable member 18. Thereby, it is unwound from the first rotatable member 13 and the first rotatable member 13 is also rotated. Furthermore, between the first and the second rotatable member 13, 18 the engagement preventing member 11 is guided by the guidance face 28 of the body 8 such that a part of the engagement preventing member 11 is visible in the window 12. In particular, one of the numbers of the engagement preventing member 11 is visible in the window 12. The number corresponds to the counted number counted by the mechanism 5. Thus, when the actuating element 54 is moved from its first position to its second position and back to its first position, the counted number is updated, e.g. incremented or decremented.

Furthermore, the guiding track 51 is configured such that the interaction member 39 is disengaged from the second rotatable member 18 close to the end of the movement of the actuating element 54 from its second position to its first position.

As can be seen in FIG. 18, the guiding element 38 of the body 8 prevents the interaction member 39 from moving out of the guiding track 51 in a direction away from the longitudinal axis x of the inhalation device 1.

The first state of the mechanism 5 corresponds to a state wherein the inhalation device 1 comprises a medical substance 2 and wherein an inhalation can be performed. In the first state, the engagement preventing member 11 covers at least partly the first surface 42 of the first rotatable member 13.

The second state of the mechanism 5 corresponds to a state wherein the inhalation device 1 is considered empty. In this state, the engagement preventing member 11 displays “0” corresponding to no doses left in the inhalation device 1. In the second state, the engagement preventing member 11 has been unwound from the first surface 42 of the second rotatable member 18 such that it covers the first surface 42 to a lower degree than in the first state.

FIGS. 20 to 29 show the mechanism 5 according to the second embodiment. As discussed above, according to the second embodiment, the mechanism 5 is additionally configured to prevent a further operation of the device 1 after a certain number of doses has been delivered. Thus, the mechanism 5 according to the second embodiment is a combined dose counting and lock-out mechanism. In another alternate embodiment, the mechanism 5 may be a lock-out mechanism which does not display the counted number.

In particular, FIGS. 20 and 21 show the body 8 of the mechanism 5 according to the second embodiment. The body 8 according to the second embodiment is constructed similar to the body 8 according to the first embodiment such that every structural and functional feature disclosed with respect to the body 8 according to the first embodiment may also be present in the body 8 according to the second embodiment.

The body 8 comprises a third pin 62 which is configured such that a blocking member 63 can be arranged on the third pin 62. The third pin 62 is arranged between the first and the second pin 30, 37. The third pin 62 defines a first rotational axis 64 wherein the blocking member 63 is configured to rotate around the first rotational axis 64. The first rotational axis 64 is parallel to the second and to the third rotational axis 16, 19.

Moreover, the first pin 30 according to the second embodiment differs from the first pin 30 according to the first embodiment as the first pin 30 comprises an engagement feature 65. The engagement feature 65 comprises an opening arranged in a sidewall of the first pin 30. The engagement feature 65 is configured to receive a blocking feature 66 of the blocking member 63. When the blocking member 63 is engaged with the engagement feature 65, the blocking member 63 is not movable relative to the body 8.

Furthermore, compared to the first embodiment, the guidance element 38 configured to prevent a movement of the interaction member 39 in a direction away from the longitudinal axis x of the inhalation device 1 is modified such that the modified guidance element 38 allows the blocking feature 66 to be moved into the guiding track 51.

Moreover, the length of the first and the second pin 30, 37 is reduced in the second embodiment. In this case, the length of the first and the second rotatable member 13, 18 is also reduced.

FIG. 22 shows a perspective view of the first rotatable member 13 according to the second embodiment. FIG. 23 shows a cross-sectional view of the first rotatable member 13 according to the second embodiment.

According to the second embodiment, the first rotatable member 13 comprises an engagement element 67. The engagement element 67 is configured to engage with the blocking member 63. The engagement element 67 comprises an opening configured to receive the blocking member 63. The engagement element 67 is arranged in the first surface 42 of the first rotatable member 13. In the first state of the mechanism 5, the first surface 42 is covered by the engagement preventing member 11. Thus, the engagement preventing member 11 is configured to prevent an engagement of the blocking member 63 and the engagement element 67 in the first state of the mechanism 5.

In a second state of the mechanism, the engagement preventing member 11 has been unwound from the first rotatable member 13 such that the engagement element 67 is free from the engagement preventing member 11. In this case, the blocking member 63 is enabled to be engaged with the engagement element 67.

Moreover, the first rotatable member 13 according to the second embodiment differs from the first rotatable member 13 according to the first embodiment as the height of the first rotatable member 13 according to the second embodiment is reduced, thereby adjusting to the reduced height of the second pin 30.

Other than the differences discussed above, the first rotatable member 13 according to the second embodiment is constructed similar to the first rotatable member 13 according to the first embodiment such that every structural and functional feature disclosed with respect to the first rotatable member 13 according to the first embodiment may also be present in the first rotatable member 13 according to the second embodiment.

Furthermore, according to the second embodiment, the mechanism 5 also comprises the second rotatable member 18. The second rotatable member 18 according to the second embodiment may differ from the second rotatable member 18 according to the first embodiment only with respect to the height. In particular, the second rotatable member 18 has a reduced height such that it is adjusted to the reduced height of the second pin 37.

Moreover, according to the second embodiment, the mechanism also comprises the engagement preventing member 11 which may be identical with the engagement preventing member 11 according to the first embodiment.

FIGS. 24 and 25 show perspective views of the blocking member 63. The blocking member 63 is configured to be arranged on the third pin 62. The blocking member 63 is configured to rotate around the first rotational axis 64.

The blocking member 63 comprises a main body 68 and the blocking feature 66. The blocking feature 66 comprises a protrusion. The blocking feature 66 extends from the main body 68 of the blocking member 63 in a direction parallel to the first rotational axis 64. The blocking feature 66 is arranged at a distance to the first rotational axis 64. Thus, when the blocking member 63 is rotated around the first rotational axis 64, the blocking feature 66 moves along a circular path around the first rotational axis 64.

Moreover, the blocking member 63 comprises a first tension member 69 and a second tension member 70. The first tension member 69 comprises an arm extending from the main body 68 of the blocking member 63. The first tension member 69 is configured to abut the first rotatable member 13 tangentially. Similarly, the second tension member 70 comprises an arm extending from the main body 68 of the blocking member 63. The second tension member 70 is configured to abut the second rotatable member 18 tangentially.

FIG. 26 shows a cross-sectional view of the inhalation device 1 comprising the mechanism 5 according to the second embodiment in the first state of the mechanism 5. The first state of the mechanism 5 corresponds to a state wherein the inhalation device 1 comprises a medical substance 2 and wherein an inhalation can be performed. In the first state, the engagement preventing member 11 covers at least partly the first surface 42 of the first rotatable member 13. In particular, the engagement preventing member 11 covers the engagement element 67 in the first state. Thus, in the first state, the blocking member 63 is not engaged with the engagement element 67. In the first state, an operation of the inhalation device 1 and of the mechanism 5 is possible.

The first and the second tension member 69, 70 abut the first and the second rotatable member 13, 18. Thereby, the blocking member 63 is tensioned such that the blocking member 63 tends to rotate around the first rotational axis 64. Furthermore, the first tension member 69 is also configured to engage the blocking member 63 with the engagement element 67 of the first rotatable member 13 and with the engagement feature 65 of the first pin 30. However, in the first state of the mechanism 5, the engagement preventing member 11 covering the engagement element 67 and the engagement feature 65 of the first pin 30 prevents the engagement of the blocking member 63 with the engagement element 67 and, respectively, with the engagement feature 65.

Moreover, in the first state of the mechanism 5, the blocking feature 66 is in a first position. The first position of the blocking feature 66 is outside the guiding track 51 such that the blocking feature 66 does not interfere with the interaction member 39 which is configured to travel along the guiding track 51.

The mechanism 5 according to the second embodiment functions in the same way as the mechanism 5 according to the first embodiment until the last dose is delivered. As more and more doses are delivered, the engagement preventing member 11 is further unwound from the first rotatable member 13 and wound around the second rotatable member 18. Thereby, the diameter of the second rotatable member 18 increases and the diameter of the first rotatable member 13 decreases. However, as the first tension member 69 abuts the first rotatable member 13 and the second tension member 70 abuts the second rotatable member 18, the blocking member 63 is rotated by a small angle around the first rotational axis 64 due to the change in the respective diameters. The tension of the blocking member 63 is not changed during this process.

FIG. 27 shows a perspective view of the inhalation device 1 comprising the mechanism 5 according to the second embodiment after the last dose has been delivered when the mechanism 5 changes from its first state to its second state. Now, the engagement preventing member 11 does not cover the engagement element 67 and the engagement feature 65 anymore. Due to the tension, the blocking member 63 is now rotated such that the blocking member 63 engages with the engagement element 67. Thereby, the blocking feature 66 is moved from its first position to its second position. In the second position, the blocking feature 66 is arranged in the guiding track 51, thereby preventing the interaction member 39 from travelling along the guiding track 51. In particular, the blocking feature 66 prevents the interaction member 39 from reaching the first position A. FIG. 27 shows the blocking feature as it is moved towards its second position.

FIG. 28 shows the mechanism 5 in its second state. Now, the blocking feature 66 is in its second position wherein it blocks a movement of the interaction member 39. The blocking member 63 is further engaged with the engagement element 67 and the engagement feature 65. Therefore, a further operation of the inhalation device 1 is prevented. In particular, it is no longer possible to carry out a dose setting operation or a dose dispensing operation as the interaction member 39 has to travel along the guiding track 51 during these operations. As these operations are now prevented, a user is warned that the device 1 is empty and no further doses can be delivered.

FIG. 29 shows a perspective view of the rotary body 25 of the inhalation device 1 comprising the clear window 12 wherein one of the numbers arranged on the engagement preventing member 11 is visible.

### REFERENCE NUMERALS

1 inhalation device
2 substance
3 housing
4 outer cylinder
5 mechanism
6 mouthpiece
7 cap
8 body
9 snap-fit element of the body
10 snap-fit element of the outer cylinder
11 engagement preventing member
12 window
13 first rotatable member
14 sub-quantity of substance
15 storage chamber
16 second rotational axis
18 second rotatable member
19 third rotational axis
20 first reverse rotation prevention feature
21 teeth of the first rotatable member
22 first pawl
23 second reverse rotation prevention feature
24 chamber sealing
25 rotary part
26 teeth of the second rotatable member
27 second pawl
28 guidance face
29 protruding flange
30 first pin
31 opening of the first rotatable member
32 first end portion
33 metering rod
34 snap fit element
35 second end portion
36 middle portion
37 second pin
38 guidance element
39 interaction member
40 metering chamber
41 second mechanism
42 first surface of the first rotatable member
43 edge
44 tool receiving opening
45 first surface of the second rotatable member
46 opening of the second rotatable member
47 edge
48 helical thread
49 first interaction feature
50 second interaction feature
51 guiding track
52 ramp
53 further guide element
54 actuating element
60 flow channel
61 intermediate channel portion
62 third pin
63 blocking member
64 first rotational axis
65 engagement feature
66 blocking feature
67 engagement element
68 main body
69 first tension member
70 second tension member
76 head
77 tongues
A first position
B second position
C track
x device axis

1. A mechanism for a drug delivery device, having a first and a second state, comprising:
an engagement preventing member, and
a blocking member comprising a blocking feature, and an engagement element which is engagable with the blocking member,
wherein the blocking member is configured to engage with the engagement element in a non-linear movement, thereby moving the blocking feature from a first position to a second position,
wherein, in the first state of the mechanism, the engagement preventing member is configured to prevent an engagement of the blocking member with the engagement element by covering the engagement element, and
wherein, in the second state of the mechanism, the engagement element is free from the engagement preventing member such that the blocking member is enabled to engage with the engagement element.

2. The mechanism according to claim 1,
wherein, when the blocking feature is in the first position, the blocking member is tensioned to rotate and thereby to move the blocking feature into the second position.

3. The mechanism according to claim 1,
wherein the blocking member is rotatable around a first rotational axis and wherein the blocking feature is offset from the first rotational axis in its first and in its second position.

4. The mechanism according to claim 1,
comprising a first rotatable member comprising the engagement element and wherein the first rotatable member is configured to be rotated by a movement of the engagement preventing member.

5. The mechanism according to claim 3,
wherein the first rotatable member is configured to rotate around a second rotational axis, and
wherein the second rotational axis is parallel to the first rotational axis.

6. The mechanism according to claim 4,
wherein the first rotatable member comprises a first reverse rotation prevention feature which is configured to permit a rotation of the first rotatable member in a first rotational direction and which is configured to prevent a rotation of the first rotatable member in a second rotational direction which is opposite to the first rotational direction.

7. The mechanism according to claim 4,
further comprising a body comprising a pin, and
wherein the first rotatable member has an opening configured to receive the pin,
wherein the first rotatable member is configured to rotate around the pin,
wherein the pin has a first end portion, a second end portion and a middle portion arranged between the first and the second end portion, and
wherein the diameter of the opening and the diameter of the pin are chosen such that the first rotatable member contacts the pin only in the first and the second end portion.

8. The mechanism according to claim 1,
comprising a second rotatable member which is configured such that the engagement preventing member is movable by a rotation of the second rotatable member.

9. The mechanism according to claim 4
wherein a first end of the engagement preventing member is attached to the first rotatable member, and
wherein a second end of the engagement preventing member is attached to the second rotatable member.

10. The mechanism according to claim 8,
wherein the engagement preventing member is wound around the first rotatable member in the first state of the mechanism and wherein the engagement preventing member is unwound from the first rotatable member by a rotation of the second rotatable member.

11. The mechanism according claim 1,
wherein the blocking member is configured to rotate when the mechanism changes from its first state to its second state.

12. The mechanism according to claim 1,
wherein the engagement preventing member comprises a tape comprising indicia configured to display information regarding the drug delivery device.

13. The mechanism according to claim 3, wherein the blocking feature is offset from the first rotational axis around which the blocking member is rotated such that the blocking feature is moved in a circular movement.

14. An assembly for a drug delivery device, comprising:
a mechanism according to claim 1, and
comprising a second mechanism comprising an interaction member configured to be moved during at least one of a dose dispensing operation and a dose setting operation of the drug delivery device,
wherein the blocking member is configured to prevent a movement of the interaction member in the second state of the mechanism, thereby preventing at least one of a dose setting operation and a dose dispensing operation of the drug delivery device.

15. The assembly according to claim 14,
comprising a guiding track wherein the interaction member moves along the guiding track during at least one of the dose setting operation and the dose dispensing operation,
wherein in its second position, the blocking feature is arranged in the guiding track, thereby blocking the movement of the interaction member along the guiding track.

16. The assembly according to claim 14,
wherein the interaction member is configured to rotate the second rotatable member when the interaction member engages with the second rotatable member.

17. The assembly according to claim 14,
wherein the second rotatable member comprises a helical thread which is configured to be engaged with the interaction member.

18. A drug delivery device, comprising:
a mechanism according to claim 1.

19. A drug delivery device, comprising:
an assembly according to claim 14.
